(19) United States
(12) Patent Application Publication
(10) Pub. No.: US 2024/0389924 A1
(43) Pub. Date: Nov. 28, 2024

(54) NEUROSCIENCE AND CONFLICT RESOLUTION

(71) Applicant: Joel Steven Goldberg, Hillsborough, NC (US)

(72) Inventor: Joel Steven Goldberg, Hillsborough, NC (US)

(21) Appl. No.: 18/797,623

(22) Filed: Aug. 8, 2024

(51) Int. Cl. A61B 5/00; A61B 5/055

(52) U.S. Cl. CPC A61B 5/4064; A61B 5/055

(57) ABSTRACT

This invention is a method to predict the motor response of presumptive negotiators after simulated decision challenges through measurement of glutamate/GABA ratios in spectral MRI (MRS) of the negotiators. The sampled voxels of MRS include precentral gyrus speech (larynx) and writing (hand) areas and/or supplementary motor cortex. The glutamate/GABA ratio or excitation/inhibition (E/I) ratio is predicted to be U shaped where an elevated E/I ratio is predicted to be impulsive decision-making and lower E/I ratio is predicted to be indecisiveness. This method may aid in the screening of negotiators who may be best suited for resolution of impasse conflicts.

### CROSS-REFERENCES TO RELATED APPLICATIONS

This application claims the benefit of U.S. application Ser. No. 14/844,202, Ser. No. 14/995,376, Ser. No. 15/057,146, Ser. No. 15/191,673, Ser. No. 16/139,062, Ser. No. 16/706,760, Ser. No. 16/924,375 and Ser. No. 17/958,328 which are incorporated by reference.

### FEDERALLY FUNDED RESEARCH

Not applicable

### BACKGROUND OF THE INVENTION

If possible, humans in civilized societies should resolve conflict without violence. Unfortunately, negotiations to resolve conflict have a poor track record with many failures.[1] In general, negotiators believe what they say and write, and in many cases they have belief systems that cannot be easily persuaded by rationality.[2] Ideologies are difficult to change.

Human behavior and the human motor system are intimately linked; however, they are both complex processes. Although there are many modulatory pathways for motor function including extrapyramidal and cerebellar pathways, the voluntary motor response is an essential component of behavior which is encoded in the action potentials of the primary motor cortex. “From a phylogenetic standpoint, voluntary motor control is extrapyramidal in sub mammalian species, mixed pyramidal/extrapyramidal in non-human mammals and essentially pyramidal in humans”[3] The motor cortex also has a decision capacity that precedes efferent signaling.[4-6] Therefore, with the exception of efferent responses from the autonomic nervous system, the human motor system is an essential determinant of behavior which is responsible for all communications including speech, writing, signs, gestures, and positions. This invention proposes to bring neuroscience from MRI spectroscopy (MRS) of the human motor cortex into the negotiation process to improve outcome.

### Prior Efforts to Include Neuroscience into the Negotiation Process

### Hyperscanning

Hyperscanning, the simultaneous measurement of brain or central nervous system (CNS) activity in dyads or groups, was discovered in 2002, and the validity of the observation has been confirmed in numerous scientific reports. Hyperscanning has provided much imaging and electrophysiological information of decision-making between dyads and among groups and may predict whether attention during conflict negotiations is directed to solving the problem cooperatively vs. competitively. With simultaneous monitoring of CNS activity during negotiations it may be possible to predict which negotiators are in synch or engaged with each other rather than preoccupied with extraneous matters during the negotiations.[7, 8]

Interbrain synchrony, which is a characteristic of cooperative decision-making, may be positively influenced by external electromagnetic fields since the chemical-electrical properties of the CNS are derived from moving charges predominantly of sodium, potassium, chloride, and calcium. The motion of these ions is not constant in velocity but accelerates as the ion channels open and close producing electromagnetic waves which is in accordance with Maxwell's equations and experimental discoveries of Hertz. These electromagnetic waves with properties of supposition and entrainment can be analyzed to sine waves through Fourier transformations.[9] Therefore, oscillations observed by such methods as EEG, which are composites of action potentials and other membrane potentials, are electromagnetic waves traveling through the body at a speed less than the speed of light. The frequency is the major determinant of information transmitted by the waves.

### Special Relativity

Conflict is an event with variants of place and time in spacetime, and past events are immutable. Special relativity and human experience teach that perception of an event and its construction in the mind may be different for observers in different reference frames. A past event that produced conflict may be interpreted differently among negotiators. Therefore, right and wrong can differ in the minds of observers not only from cultural bias, but from the variant information from an event. Passage of time changes the nature of a conflict, and time may be the negotiators' best friend.

### Second Law of Thermodynamics

Conflict is an event with a temporal component. As the arrow of time increases entropy, so must the conflict change. Therefore, it is highly unlikely that any future interventions can restore the conflict to its original status, which may be the explanation that reparations may not lead to resolution of a conflict.

### Game Theory

Win-win resolution of a conflict is the preferred outcome in which both parties receive more than expected, and further negotiations are unlikely to improve the outcome for either side. Win-win resolutions often require creativity and “out of the box” thinking, patience, and perseverance. Game theory teaches that a lose-lose outcome or compromise may be

transformed into (lose-lose)-win which is a more favorable outcome for conflict resolution.[10] The win is not arbitration or mediation, but an economic entity, either tangible or intangible that contributes to both parties and cushions the feelings of loss.

### Geomagnetic and Seasonal Influences

It is well known that seasonality influences the incidence of psychiatric conditions including psychiatric hospitalizations.[11] The earth's magnetic field changes with events such as solar storms including coronal mass injections, penetrating cosmic rays, and inherent variability which also may affect health.[12, 13] Magnetic field penetration into the central nervous system is well established, but its effects on behavior in the micro tesla range are controversial. Independent of these effects are rapid changes in daylight that affect circadian rhythm and Schumann resonance effects, both of which may affect mood.[14, 15]

### Neuroeconomics

In many cultures, economic structure is the foundation of other geographic, political, religious, and social structures. There are economic concerns in nearly every conflict, some of which may be divisible for resolution of the conflict. If the conflict involves a divisible attribute, negotiating within the bargaining range may be preferable to resolving the conflict with force.[16] Understanding anchoring, references points, sunk costs, and loss aversion as it applies to the conflict may be essential for resolution. Present macroeconomic policies that favor inflation complicate economic conflicts because inflation includes unpredictable future value.

### Psychology

Negotiators may not make decisions based upon deliberated rationality, but rather

resort to heuristic System 1 solutions which most likely will yield inferior results.[17] A three dimensional model of interactive human behavior which claims to predict behavior among groups or dyads suggests that behaviors and relationships are correlated based on culture.[18]

### Global Stress Events

The effects of a global traumatic event on conflict resolution can only be hypothesized, but need to be considered since such events are clearly within the range of possibility. A nuclear war, asteroid collision with earth, alien contact, climate crisis, crippling electromagnetic pulse, crippling cyberattack, and devastating pandemic are just some of the projected possibilities.[19] How the world managed the COVID crisis showed that world unity was lacking, and sovereign states chose to manage the crisis with primarily self-interest. How these possible global traumatic events would impair the human mind and behavior will affect conflict resolution, but it could be a positive effect if unity of purpose emerges from sovereign states.

### Predicting Negotiator Behavior from MRS

MRS of the brain can non-invasively reveal chemical makeup of voxels of brain tissue. Common measurements include N Acetyl aspartate (NAA), Choline (CH), Glutamate (GLU), Glutamine (GLN), Gamma aminobutyric acid (GABA), Creatine (CR), and Myoinositol (ml). These measurements and their ratios enable one to discriminate many brain tissue diseases and psychiatric diseases including schizophrenia, bipolar, posttraumatic stress, and obsessive-compulsive disorders. As technology progresses with greater discriminative sampling, particularly of the motor system of the brain, profiles of behavior can be predicted. These profiles can aid in the selection of suitable negotiators, which is the focus of this invention. These negotiators may not be the most educated, intelligent, or politically chosen choices.

### PRIOR ART

There are no known references citing motor cortex MRS excitation/inhibition ratio or glutamate/GABA ratio as a determinant of decision-making in negotiators.

The following references cite MRS or transcranial magnetic stimulation (TMS) excitation/inhibition ratio or glutamate/GABA ratio in the primary motor cortex, supplementary motor cortex, or prefrontal cortex as a determinant of decisions:
1. (Supplementary Motor) Cortical glutamate and GABA are related to compulsive behavior in individuals with obsessive compulsive disorder and healthy controls[20]
2. (Prefrontal) Dissociable roles of cortical excitation-inhibition balance during patch-leaving versus value-guided decisions[21]
3. Functional Changes in GABA and Glutamate during Motor Learning[22]
4. (TMS) Deficient GABAergic and glutamatergic excitability in the motor cortex of patients with long-COVID and cognitive impairment[23]
5. Cognitive control affects motor learning through local variations in GABA within the primary motor cortex[24]
6. (In Vitro) Effects of altered Excitation-Inhibition Balance on Decision Making in a Cortical Circuit Model[25]
7. (Review) GABA, Glutamate and Neural Activity: A Systemic Review with Met-Analysis of Multimodal ¹H-MRS-fMRI Studies[26]

The following references cite MRS excitation/inhibition ratio or glutamate/GABA ratio in the primary motor cortex without a relationship to decision-making:
1. Short echo-time Magnetic Resonance Spectroscopy in ALS, simultaneous quantification of glutamate and GABA at 3T[27]
2. Quantification of GABA, glutamate, and glutamine in a single measurement at 3T using GABA-edited MEGA-PRESS[28]
3. Relationship between physiologic measures of excitability and levels of glutamate and GABA in the human motor cortex[29]

The following reference cites GABA, but not glutamate MRS in the supplementary motor cortex and decision-making:

Individual differences in subconscious motor control predicted by GABA concentration in SMA[30]

The following reference cites MRS glutamate, but not GABA in the motor cortex as a determinant of decisions:

Increased Glutamate concentrations during prolonged motor activation as measured using functional Magnetic Resonance Spectroscopy at 3T[31]

### BRIEF DESCRIPTION OF THE DRAWINGS

Not applicable

### BRIEF SUMMARY OF THE INVENTION

This invention is a method to predict motor responses of negotiators through analysis of MRS of the human motor system which is intimately linked to behavior. The excitatory/inhibitory ratio best expressed as the glutamate/GABA ratio of motor neurons predicts this activity. This measurement can be performed before selecting negotiators for their assigned task, and it can be performed multiple times after a simulation decision challenge. Application of this methodology may prove useful for selecting negotiators for resolution of impasse conflicts.

### DETAILED DESCRIPTION OF THE INVENTION

All living organisms exist in an open non-equilibrium thermodynamic system in which the chemistry of ion fluxes, predominately through fluxes of sodium, potassium, calcium, and chloride, determines the electrical properties. These fluxes are either excitatory or inhibitor, and the two most abundant neurotransmitters glutamate and GABA are most influential. “The synaptic balance between excitation and inhibition (E/I balance) is a fundamental principle of cortical circuits, and disruptions in the E/I balance are commonly linked to cognitive deficits such as impaired decision-making”.[25] It is now possible to non-invasively through MRS determine the glutamate/GABA in voxels of motor neurons in the brain.

At the present time, understanding how distinct areas of the brain produce motor system changes is not possible. The circuitry is quite complex. However, understanding the excitatory/inhibitory ratio of motor neurons during decision-making may aid in the selection of negotiators who have the optimum characteristics to resolve impasse negotiations.

The glutamate/GABA ratio is well recognized as a marker of the excitation/inhibition ratio in neural activity, and the glutamate/GABA ratio has been measured in decision-making mostly in non-motor sampling. Elevated excitation/inhibition ratio or glutamate/GABA ratio in motor neurons may profile a negotiator who is impulsive and lowered excitation/inhibition ratio or glutamate/GABA ratio may profile a negotiator who is indecisive.[25] Furthermore, it is hypothesized that negotiators who are at peace with themselves are most likely to be able to interact with their counterpart with empathy, which optimizes the likelihood of a satisfactory resolution, especially when there are challenges to belief systems.[32]

### Glutamate is the Major Excitatory Transmitter

Glutamate is the major excitatory transmitter. It is a link between chemistry and electrical properties in the nervous system. Glutamate via alpha keto glutamate, an intermediary in the Krebs cycle, is a bridge between energy production via Krebs cycle and nervous system activity. The effects of glutamate are on both ionotropic and metabotropic receptors, the former being the most direct influence of sodium flux through NMDA, AMPA, and kainite receptors, and the latter being a G protein-coupled receptor (GPCR) function.

Glutamate does not cross the blood-brain barrier (BBB), and two other sources of glutamate for neurons include: 1) glutamine from glial cells and 2) glutamine from plasma. However, most investigators agree that the major source of intraneural cytoplasmic glutamate is from the Krebs cycle. Although there may be other excitatory amino acid transmitters such as aspartate, cysteine, and homocysteine, their role is generally accepted as minor compared to glutamate. Glutamate is easily and commonly detected by MRS at 2.2-2.4 ppm.

### GABA is the Major Inhibitory Transmitter

GABA is primarily derived from a-decarboxylation of L-glutamic acid catalyzed by glutamic acid decarboxylase (GAD), and GABA does not cross the BBB. The inotropic GABA_{A} receptor, which regulates passage of chloride ions is an important inhibitory receptor which hyperpolarizes neurons. GABA is linked to the Krebs cycle through transamination by GABA transaminase (GABA-T), but this reaction is not known to be a major source of GABA. GABA can be measured with some difficulty with 7 tesla MRS since its concentration in cortical regions is ˜1 mmol/L. GABA is best detected with J coupling of the 3.0 ppm GABA resonance with the GABA resonance at 1.9 ppm to obtain the GABA multiplet at the 3.0 ppm chemical shift position without overlap with other resonances.[33]

### Motor Cortex

The motor cortex of the precentral gyrus is represented by Brodmann area 4, and the areas of particular interest to sample by MRS are those representing speech and writing which are basic activities of negotiators and/or the supplementary motor cortex. The writing area of the motor cortex is best represented in the hand knob region of the hemispheres.[34, 35] The motor supply to the larynx is in Brodman area 4. “The precentral gyrus or area 4 initiates all voluntary movements.”[36] This sampling assumes that all language areas such as Broca and Wernicke areas, extrapyramidal pathways, basal ganglia, and cerebellar function are intact.

### Protocol for Determination of Glutamate/GABA ratio in Presumptive Negotiators

Presumptive negotiators are screened in a 7 tesla MRI machine. Obtaining optimum glutamate and GABA concentrations and glutamate/GABA ratios will require shimming to homogenize the Bo field, correcting for chemical shifting displacement error, correcting for water and lipid spectra, pulsating with either PRESS (point-resolved spectroscopy), STEAM (stimulated echo acquisition mode), or LASER (localization by adiabatic selective refocusing), and determining empirically echo time (TE). These data are compared to a population of normal controls. After baseline glutamate/GABA ratios are obtained, the presumptive negotiators are presented with a decision challenge simulating the conflict which they are intended to negotiate, and they are rescanned to determine their glutamate/GABA ratio in the motor regions of interest corresponding to hand and speech and/or supplementary motor area, which is processed to predict their behavior during conflict negotiations. Simulations are commonly used in negotiation training. This information will guide in the selection of negotiators for the conflict.

### Utility of this Invention

Conflict negotiations, to include peace negotiations, have a projected poor outcome. Some of the reasons for this inadequacy include:
1. Failure of Preemptive Efforts

Once conflict is identified, efforts should be made to prevent escalation prior to use of force. To allow conflicts to smolder over time is an action that is unlikely to improve the situation and may lead to rapid decisions that escalate to violence. Sufficient resources need to be allocated by sponsors at this early stage of conflict, and funding will often pay off handsomely over time. At this early stage, time is an asset as it has been shown in neuroscience that thoughtful decisions (System 2) may produce a more favorable outcome than rapid heuristic responses (System 1).[17]
2. Failure of Trust

Conflicts are resolved without violence through cooperative mutual understanding and trust. Without trust, human society could not exist.
3. Failure to Understand the Other Side

Neuroscience teaches that most individuals believe what they say and write. It is crucial that negotiators spend considerable resources and time to understand the adversary. Science and reality teach us that perceptions of events differ considerably. Beliefs are embedded in a conflict, and it may be very difficult to change beliefs with facts.[2] These beliefs have evolved over time from past experiences, which are different for different observers. One needs to study the other side as intently as one studies your side.
4. Failure of Empathy

Some studies suggest that empathy during negotiations may produce positive outcomes, and it is often lacking in negotiations.[37] Empathy may address the dichotomy between beliefs and rationalism. Beliefs are so ingrained in the human psyche that facts may not change them, and an attempt to change these beliefs may be futile since the mind will reconstruct the facts in numerous ways to support their beliefs. This is particularly true when beliefs are faith based and deeply ingrained in culture. Empathy may be especially useful where moral arguments are part of the conflict, and there is participant dehumanization. Empathy may help resolve what seems to be intractable.
5. Failure to Resolve Divisible Economic Issues

Nearly all conflicts involve economics such that property is contested, and it is most often territory in international conflicts. In most instances, such property is divisible (cutting the pie), but unless the property can be enhanced (bigger pie), most divisions will require compromise, a lose-lose outcome, and unlikely a win-win. The probability of gain from a violent resolution from a divisible property conflict has been shown to be, in many instances, inferior to a negotiated outcome. The bargaining range most often exceeds the rewards from a forced, especially armed conflict, where inevitable losses occur.[16]
6. Failure to Reframe the Conflict Utilizing Time

The passage of time reframes every conflict. It must do so, since a conflict is an event and defined in spacetime in which time is a variant. Issues that are of paramount importance in one time frame may become of lesser concern in a different reference frame. Incremental steps can be important, and time also permits conditional resolutions.
7. Failure to include all Parties in Conflict

Some of the biggest mistakes in international negotiation history which produced poor outcome have excluded lower power participants. The prime example is the 1947 UN partition of Palestine where a majority vote determined the decision. Striving for maximum consensus is often preferable to settling for majority vote. It is impossible for all voices to be heard in international multifactorial conflict, but every effort needs to be made to ensure representation of as many groups as possible. George Mitchell and his team in negotiating Northern Ireland peace were careful to include representatives from all parties, and that action may have been a factor for the success of the negotiations.[38] After the peace was negotiated and the documents were signed, a referendum followed.
8. Failure to Appreciate the Potential Benefits of Gender Diversity

There is some evidence that diversity, especially gender diversity, may improve conflict resolution. For centuries women have been excluded from high stakes conflict negotiations, but the female experience brings to the negotiation table resources lacking in an all-male conflict.
9. Failure to Utilize the Benefits of Multi-track Negotiations in International Conflicts

When prominent individuals negotiate, it is rarely a private discussion, and the media and political implications taint the negotiation process, which is amplified in our interconnected world of information dissemination. Behind the scenes resolution work can experiment with novel ways to resolve conflict which is the focus of this invention. Neuroscience has progressed such that we better understand the cooperative decision process, and novel interventions may prove fruitful, especially when stalemate or an impasse has been reached. Unless we try, we will not know if these interventions will aid in conflict, but the failure rate of some negotiations is so high that it would be unwise not to try.
10. Failure to Appreciate Different Perceptions of Past Events

In any negotiation, the past can become a focal point of conflict. Each side has its own grievances related to what they perceive as past wrongdoings and may stall or stalemate the negotiation process. As difficult as it may seem, retribution needs to be segregated from the conflict resolution process by addressing the present. The present and future are what matters most.
11. Failure to Effectively Utilize Deterrence

Deterrence has been shown to be an effective action to check aggression in conflict, and the success of deterrence is related to the motivation of the aggressor. The concept of “firm but flexible” has been advocated to improve the probabilities that deterrence can prevent war. Although several factors influence whether deterrence will prevent war, including balance of power and unambiguous rhetoric and action, probably the motivation of a perceived aggressor is the most important factor that determines the effectiveness of deterrence.
12. Failure to Appreciate Resolution vs Reconciliation of Conflict

Reconciliation is a process that occurs in the mind which may require generations to achieve and may be associated with forgiveness. However, it is not required for resolution of conflict.[39] Forgiveness for perceived wrongdoing is addressed differently in different corporate organizations, governments, cultures, and religions. Reconciliation is a long and arduous process that occurs in the mind.
13. Failure to Appreciate Communication Difficulties

A common language was felt to improve the chances of peace negotiations. In 1887 L. L. Zamenhof, founder of Esperanto, recognized that direct communication would be beneficial for the peace process and created Esperanto with this in mind. He was also nominated multiple times for the Nobel Peace Prize for this work. Face-to-face communication has been shown to be more effective than remote communication; however, interpreters who are essential for international communication change normal face-to-face communication.
14. Failure of Independent Judgements

Group or team decisions are often skewed from objectivity, especially when consensus is the System 1 heuristic decision. Independent thinking most often improves the decision-making process, and those in the group who speak assertively and early often sway the opinions of others.
15. Failure to Utilize Artificial Intelligence

Artificial intelligence algorithms may be extremely useful for discovering multiple decision-making options.
16. International Conflict Resolution

Conflict is a natural part of history and inevitable among countries and their leaders and constituents. When conflicts cannot be peacefully negotiated, force may become the method to resolve the disputes.

Going to war to settle conflict is associated with significant risk. No matter how careful the plan and how abundant are the resources, leaders and their constituents take risk, and the outcomes are far from certain. Numerous examples in recent world history support this proposition. The Nazi invasion of Russia in WWII, US deployments in Vietnam and Afghanistan, 1982 Israel invasion of Lebanon, Iraq invasion of Kuwait, and Russian invasion of Ukraine were not short and easily won wars. Risk taking is the composite of the emotional, cognitive, and physiologic processes in the minds of the leaders. When leaders become paranoid, megalomaniacal, impulsive, and narcissistic and possess the machinery to win a war, they will often undertake the risk of war.

The environment has effects on the mind. Many peace negotiations have been formally conducted in “neutral” environments such as Oslo, Malta, and Geneva. Some peace negotiations have been conducted in symbolic locations such as Gettysburg or in informal locations such as the Reagan ranch. Whether the negotiations are formal or informal, the meeting of the minds is influenced by the environment.

The above are some of the reasons conflict resolutions often fail. It is not a complete list, and every conflict has unique participants, goals, and outcomes. There is no blueprint for conflict resolution. All conflicts are different with different participants.

### REFERENCES

### Addin En.Reflist

1. Faure, G.O., Unfinished Business Why International Negotiations Fail. 2012, Athens, Georgia: University of Georgia Press.
2. Fitzduff, M., Our brains at war: the neuroscience of conflict and peacebuilding. 2021, New York, NY: Oxford University Press,.
3. Campbell, W.W., DeJong's The Neurologic Examiniation. 2005, Philadelphia, PA: LIPPINCOTT WILLIAMS & WILKINS.
4. Eisenstein, T., E. Furman-Haran, and A. Tal, Early excitatory-inhibitory cortical modifications following skill learning are associated with motor memory consolidation and plasticity overnight. Nat Commun, 2024. 15(1).
5. Heekeren, H.R., S. Marrett, and L.G. Ungerleider, The neural systems that mediate human perceptual decision making. Nat Rev Neurosci, 2008. 9(6): p. 467-79.
6. Hanks, T.D. and C. Summerfield, Perceptual Decision Making in Rodents, Monkeys, and Humans. Neuron, 2017. 93(1): p. 15-31.
7. Goldberg, J.S., Jackson, C. L., METHOD TO IMPROVE OUTCOMES DURING NEGOTIATIONS US 2020/0108264 A1.
8. Goldberg, J.S., OXYGEN REDUCTION REACTIONS AND INTERBRAIN SYNCHRONY US 2023/0041085 A1.
9. Goldberg, J.S., REGULATION OF THALAMIC ACTIVITY FOR CONTROL OF AGGRESSION US 2019/0030335 A1.
10. Goldberg, J.S., (LOSE-LOSE)-WIN RESOLUTION OF CONFLICT US 2020/0349667 A1.
11. Zhang, R. and N.D. Volkow, Seasonality of brain function: role in psychiatric disorders. Transl Psychiatry, 2023. 13(1): p. 1-11.
12. Feigin, V.L., et al., Geomagnetic storms can trigger stroke: evidence from 6 large population-based studies in Europe and Australasia. Stroke, 2014. 45(6): p. 1639-45.
13. Kay, R.W., Geomagnetic storms: association with incidence of depression as measured by hospital admission. Br J Psychiatry, 1994. 164(3): p. 403-9.
14. Goldberg, J.S., THERMODYNAMIC MODEL OF A NERVOUS SYSTEM US 2015/0379898 A1.
15. Goldberg, J.S., METHOD TO OPTIMIZE ELECTRODE PLACEMENT FOR CRANIAL ELECTRICAL STIMULATION US 2016/0129238 A1.
16. Fearon, J.D., Rationalist Explanation for War. International Organization, 1995. 49(3): p. 379-414.
17. Kahneman, D., Thinking, fast and slow. 1st ed. 2011, New York: Farrar, Straus and Giroux.
18. Pak, M., A Three-Dimensional Model of Human Behavior. 2004, Bloomington, IN: 1stBooks Publishing Co., Inc.
19. Goldberg, J.S., Global Traumatic Stress: Hypothetical Events and Possible Solutions. Traumatology, 2009. 15: p. 78-80.
20. Biria, M., et al., Cortical glutamate and GABA are related to compulsive behaviour in individuals with obsessive compulsive disorder and healthy controls. Nat Commun, 2023. 14(1): p. 3324.
21. Kaiser, L.F., et al., Dissociable roles of cortical excitation-inhibition balance during patch-leaving versus value-guided decisions. Nat Commun, 2021. 12(1): p. 1-13.
22. Bell, T.K., et al., Functional Changes in GABA and Glutamate during Motor Learning. eNeuro, 2023. 10 (2).
23. Manganotti, P., et al., Deficient GABABergic and glutamatergic excitability in the motor cortex of patients with long-COVID and cognitive impairment. Clin Neurophysiol, 2023. 151: p. 83-91.
24. Maruyama, S., et al., Cognitive control affects motor learning through local variations in GABA within the primary motor cortex. Sci Rep, 2021. 11(1): p. 18566.
25. Lam, N.H., et al., Effects of Altered Excitation-Inhibition Balance on Decision Making in a Cortical Circuit Model. J Neurosci, 2022. 42(6): p. 1035-1053.
26. Kiemes, A., et al., GABA, Glutamate and Neural Activity: A Systematic Review With Meta-Analysis of Multimodal (1)H-MRS-fMRI Studies. Front Psychiatry, 2021. 12.
27. Blicher, J.U., et al., Short echo-time Magnetic Resonance Spectroscopy in ALS, simultaneous quantification of glutamate and GABA at 3 T. Sci Rep, 2019. 9(1): p. 17593.
28. Sanaei Nezhad, F., et al., Quantification of GABA, glutamate and glutamine in a single measurement at 3 T using GABA-edited MEGA-PRESS. NMR Biomed, 2018. 31(1).
29. Stagg, C.J., et al., Relationship between physiological measures of excitability and levels of glutamate and GABA in the human motor cortex. J Physiol, 2011. 589(Pt 23): p. 5845-55.
30. Boy, F., et al., Individual differences in subconscious motor control predicted by GABA concentration in SMA. Curr Biol, 2010. 20(19): p. 1779-85.
31. Volovyk, O. and A. Tal, Increased Glutamate concentrations during prolonged motor activation as measured using functional Magnetic Resonance Spectroscopy at 3T. Neuroimage, 2020. 223: p. 117338.
32. Goldberg, J.S., METHOD TO MAINTAIN PEACE THROUGH ELECTROMAGNETIC ENERGY TARGETED TO THE BRAIN US 2016/0375220 A1.
33. Öz, G., Magnetic Resonance Spectroscopy of Degenerative Brain Diseases. 2016, Switzerland: Springer.
34. Yousry, T.A., et al., Localization of the motor hand area to a knob on the precentral gyrus. A new landmark. Brain, 1997. 120 (Pt 1): p. 141-57.
35. Caulo, M., et al., New morphologic variants of the hand motor cortex as seen with MR imaging in a large study population. AJNR Am J Neuroradiol, 2007. 28(8): p. 1480-5.
36. Duus, P., Topical Diagnosis in Neurology. 1989, New York, N.Y.: Thieme Medical Publishers, Inc.
37 Bruneau, E.G., M. Cikara, and R. Saxe, Parochial Empathy Predicts Reduced Altruism and the Endorsement of Passive Harm. Soc Psychol Personal Sci, 2017. 8(8): p. 934-942.
38. Mitchell, G.J., The Negotiator. 2015, New York, N.Y.: Simon & Schuster
39. Goldberg, J.S., COHERENT ELECTROMAGNETIC WAVES AID RECONCILIATION US 2016/0302667 A1.

Having described my invention, I claim:

1. A method of predicting a motor response in a negotiator comprising:
(a) measuring with a magnetic resonance imaging device a concentration of glutamate and gamma aminobutyric acid (GABA) within a voxel of interest in a primary motor cortex of the negotiator;
(b) challenging the negotiator of step (a) with a simulation of a conflict;
(c) repeating step (a);
(d) processing the glutamate and GABA concentration of steps (a) and (c) as a ratio; and
(e) predicting the motor response of the negotiator from the ratio of step (d).

2. A method of predicting a motor response in a negotiator comprising:
(a) measuring with a magnetic resonance imaging device a concentration of glutamate and gamma aminobutyric acid (GABA) within a voxel of interest in a supplementary motor cortex of the negotiator;
(b) challenging the negotiator of step (a) with a simulation of a conflict;
(c) repeating step (a);
(d) processing the glutamate and GABA concentration of steps (a) and (c) as a ratio; and
(e) predicting the motor response of the negotiator from the ratio of step (d).
